(19) United States
(12) Patent Application Publication
(10) Pub. No.: US 2005/0119667 A1
(43) Pub. Date: Jun. 2, 2005

(54) Clip-type surgical instrument for spinal implant

(75) Inventors: Tiphaine Leport, Lille (FR); Jean-Yves Leroy, Campagne Les Hesdin (FR); Guy Viart, Saint-Leger (FR); Pascal Rokegem, Arras (FR)

Correspondence Address: YOUNG & THOMPSON, 745 SOUTH 23RD STREET, 2ND FLOOR, ARLINGTON, VA, 22202, US

(73) Assignee: EUROSURGICAL SA, BEAURAINS (FR)

(21) Appl. No.: 10/898,320

(22) Filed: Jul. 26, 2004

(30) Foreign Application Priority Data
Dec. 2, 2003 (FR) 03 14130

(51) Int. Cl. A61B 17/56

(52) U.S. Cl. 606/104; 606/61; 606/72

(57) ABSTRACT

The surgical instrument according to the present invention comprises a main body (101) comprising a horizontal tube (102) extending by a frame (103) that is formed as an integral unit with a fixed handle (104), a movable handle (105) acting on a driving device (108) guided within the frame (103) and allowing, according to the pressure force applied on the movable handle (105), firstly the immobilization of the surgical instrument (100) on the head (8) of the bone anchoring element (2) and secondly the introduction under the pushing force F of an immobilizing element (104), previously positioned and guided within the tube (102), in the head (8) of the bone anchoring element (2).

The present invention relates to a clip-type surgical instrument allowing the introduction and positioning of an immobilizing element in the head of a bone anchoring element that is a member of a spinal implant.

The surgical instrument according to the present invention is more particularly designed for spinal implants comprising a bone anchoring element comprising a U-shaped head provided to cooperate with an immobilizing element allowing through a tightening screw the immobilization in translation and rotation of a connection rod, interconnecting each bone anchoring element.

The surgical instrument according to the present invention is provided for improving and facilitating the introduction of each immobilizing element within the U-shaped head of the corresponding bone anchoring element.

The surgical instrument according to the present invention comprises a main body comprising a horizontal tube extending by a frame forming an integral unit with a fixed handle, a movable handle acting on a driving device guided within the frame and allowing, according to the pressure force applied on the movable handle on the one hand, the immobilization of the surgical instrument on the head of a bone anchoring element and on the other hand, the introduction under a pushing force F of an immobilizing element, previously positioned and guided within the tube, in the head of the bone anchoring element.

The surgical instrument according to the present invention comprises a movable handle that cooperates with the fixed handle through an axis of rotation and a laminated spring allowing the driving device to be activated under a pressure force and to return to its position of rest after releasing the pressure force.

The surgical instrument according to the present invention comprises a movable handle that comprises above the axis of rotation a head presenting a domed profile from which a fork extends wherein each branch comprises a free extremity at the rounded profile.

The surgical instrument according to the present invention comprises a frame that is comprised of two walls, i.e., a vertical wall and an opposite wall interconnected by upper and lower perforated walls, said vertical wall being formed as an integral unit with the horizontal tube presenting an internal bore opening within the frame, while the opposite wall is pierced by a hole carried by the same main axes as that of the internal bore, said upper wall presenting a central rectangular-shaped recess and an extension extending beyond the vertical wall and in which an oblong groove is pierced, while the lower wall comprises another recess disposed below the latter, said recess comprising internal shoulders designed to form a stop at the fixed handle.

The surgical instrument according to the present invention comprises a locking tube comprising at one of its extremities fork-shaped branches, each notched so that the free extremities of each branch of the movable handle cooperate respectively with said notches to allow under the action of the movable handle the sliding of the locking tube around the tube, and at the opposite extremity a cylindrical sleeve extending by parallel tabs.

The surgical instrument according to the present invention comprises a cylindrical plunger that crosses the frame through the hole provided in the vertical wall to slide in the internal bore of the tube.

The surgical instrument according to the present invention comprises a driving device that is comprised of a first plate elastically loaded by a spring and disposed within the frame so as to cooperate with the recesses of the upper and lower walls and the cylindrical plunger and of a second plate elastically loaded by a spring and disposed within the extension groove of the upper wall so as to cooperate with the cylindrical plunger, said first plate being provided for bearing, under the force of the spring, against the domed profile of the movable handle head allowing during the activation of the latter the displacement in translation of the cylindrical plunger within the tube, due to the straight position of the first plate.

The surgical instrument according to the present invention comprises a tube that comprises in the extension of the cylindrical internal bore and at its free extremity, another internal bore wherein the internal profile is complementary to the external profile of the immobilizing element in order to be able to receive and guide the latter before its introduction in the head of the bone anchoring element.

The surgical instrument according to the present invention comprises a cylindrical tube that is extended at its free extremity by a roughly rectangular sleeve wherein each parallel and opposite wall comprises a U-shaped cutout designed to cooperate with the connection rod of the spinal implant.

The surgical instrument according to the present invention comprises a cylindrical tube wherein the internal profile of the sleeve is measured in slightly greater dimensions than those of the internal bore in order to conform the shoulders forming a stop to the input of the latter.

The surgical instrument according to the present invention comprises a cylindrical tube that comprises on its external profile flat parts that are extended on each wall of the sleeve, each flat part being pierced at the tube of the first and second holes opening within the bore and at the walls of the sleeve of a third hole opening.

The surgical instrument according to the present invention comprises a cylindrical tube wherein each first hole presents an internal threading allowing it to cooperate with a tightening screw for fixing two elastic strips in each flat part.

The surgical instrument according to the present invention comprises a cylindrical tube wherein each elastic strip is formed as an integral unit with a pin cooperating with the second corresponding hole and within the bore to allow the immobilizing elements to be held within the latter.

The surgical instrument according to the present invention comprises a cylindrical tube wherein each elastic strip is formed as an integral unit with a pin cooperating with the third hole of the sleeve in order to be able to penetrate within the latter when the strips are deformed by an external pushing force coming from the tabs of the locking tube in order to allow the surgical instrument to be fixed on the head of the bone anchoring element.

The description that is going to follow with regards to the attached drawings is given by way of non-limiting example, and will allow the invention, the characteristics that the invention presents and the advantages that the invention is likely to bring to be better understood:

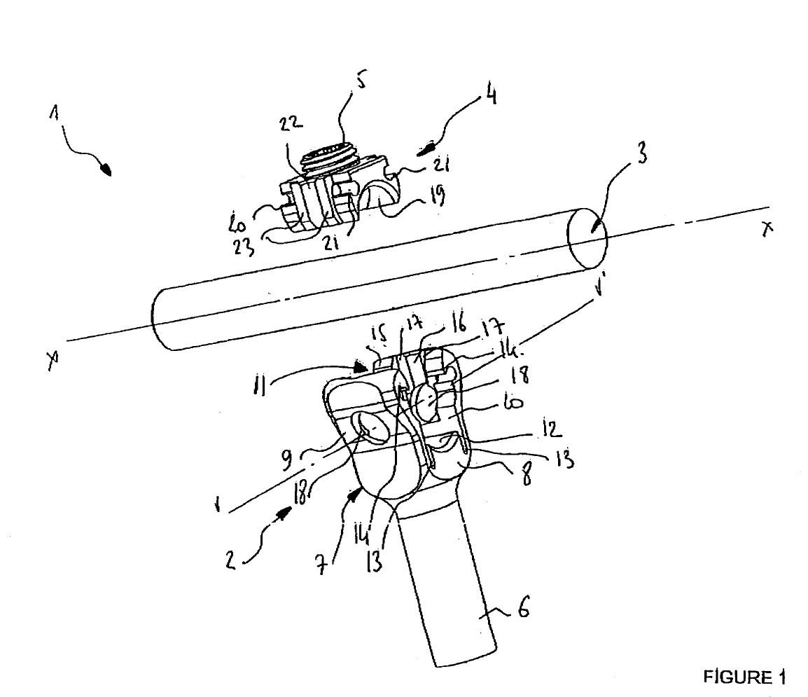

FIG. 1 is an exploded perspective view illustrating for example a bone anchoring element of a spinal implant for which the surgical instrument according to the present invention may be utilized.

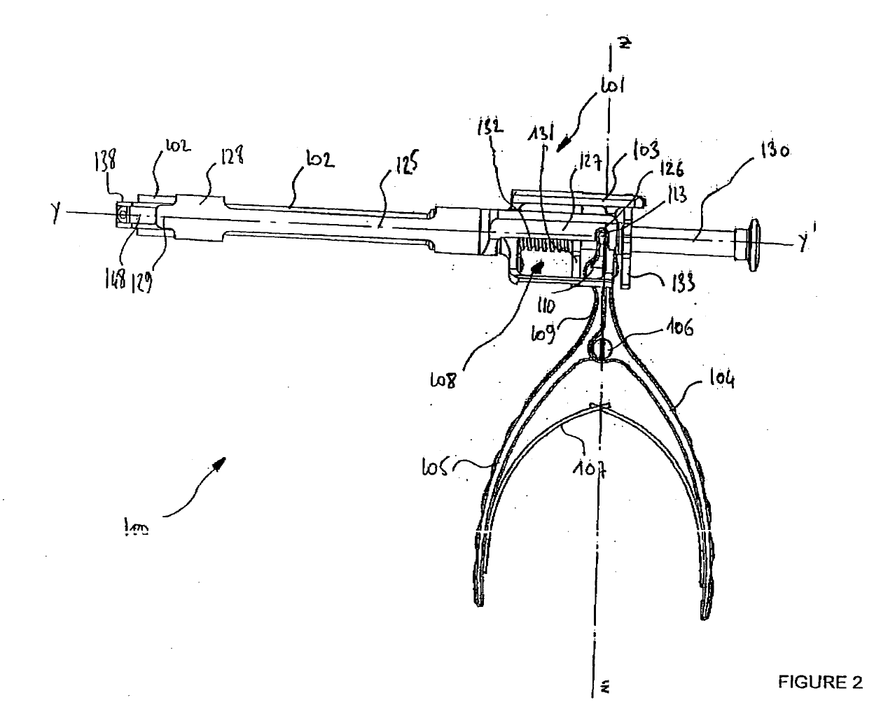

FIG. 2 is a view representing the surgical instrument according to the present invention.

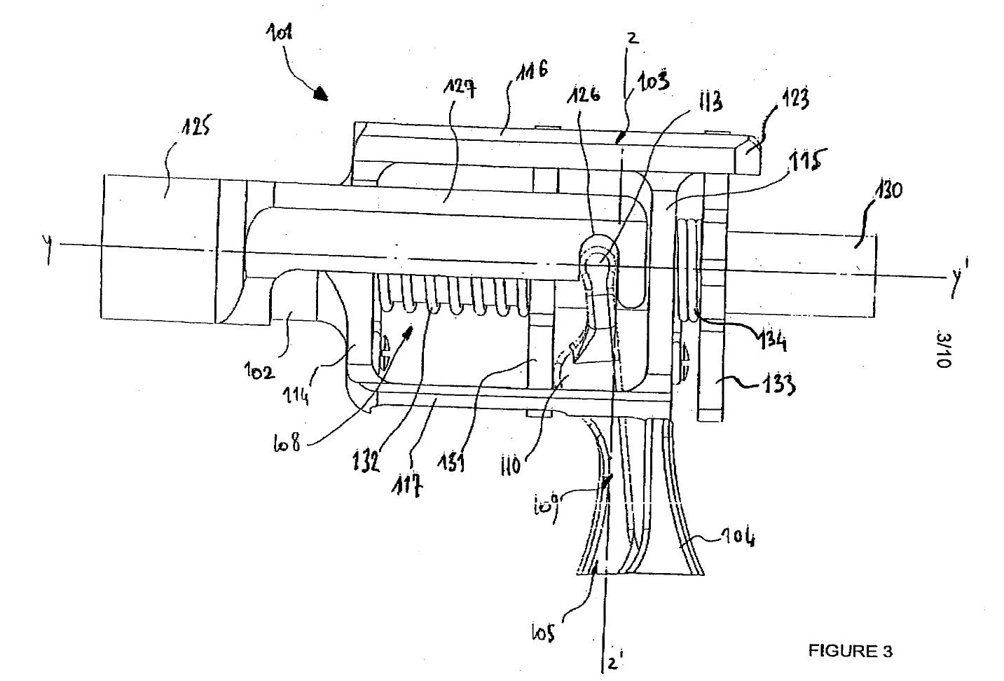

FIG. 3 is a detailed view showing the driving device of the surgical instrument according to the present invention.

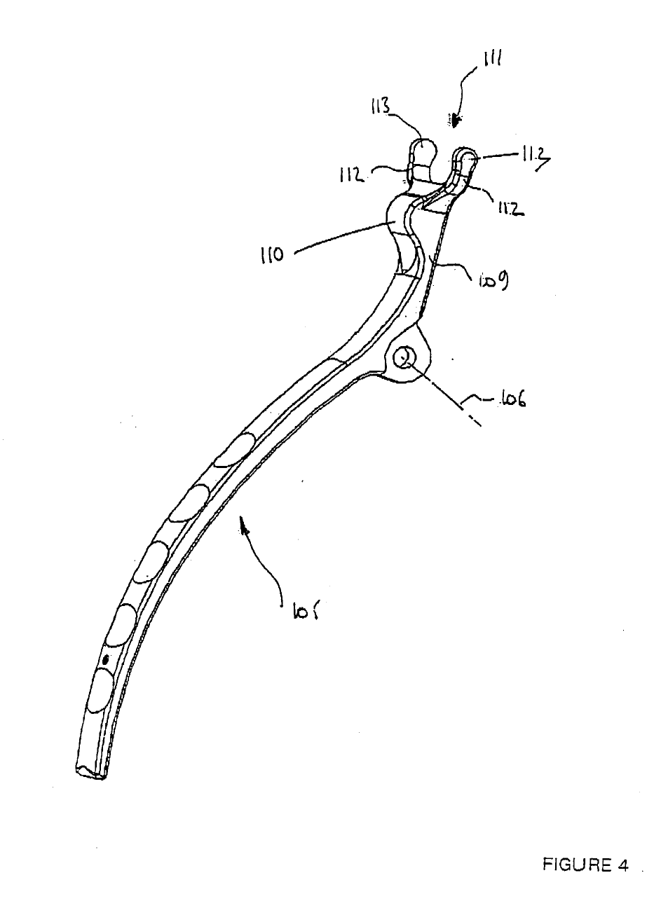

FIG. 4 is a perspective view illustrating the movable handle of the surgical instrument according to the present invention.

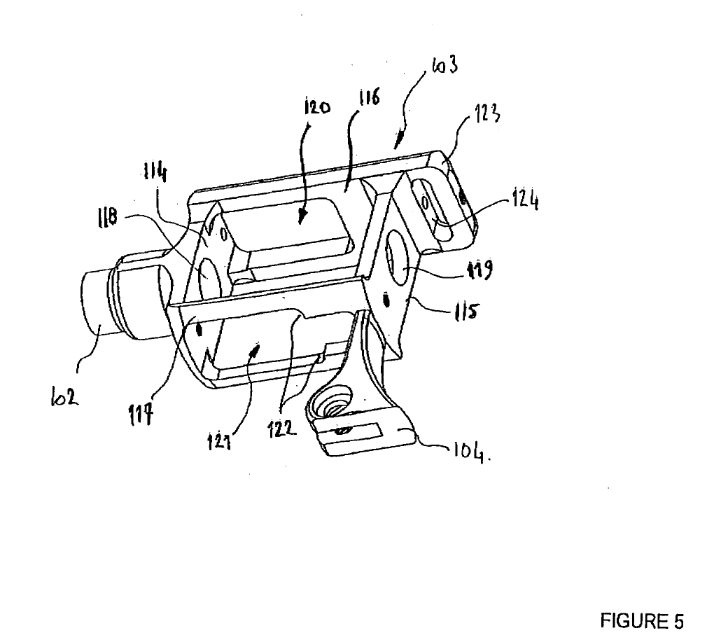

FIG. 5 is a perspective view showing the frame of the main body of the surgical instrument according to the present invention.

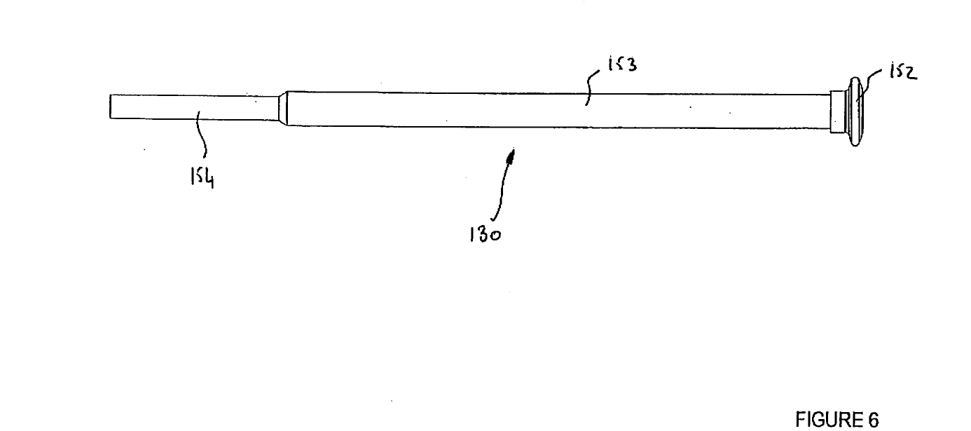

FIG. 6 is a view illustrating the cylindrical plunger of the surgical instrument according to the present invention.

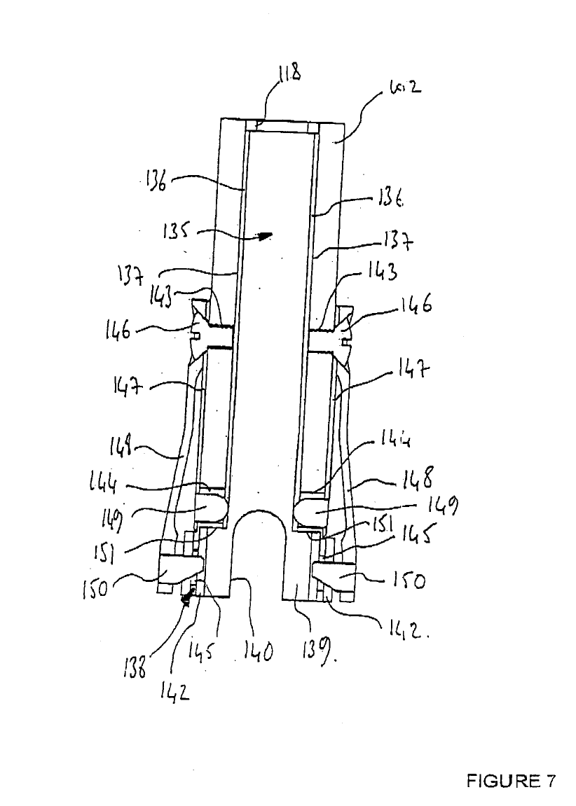

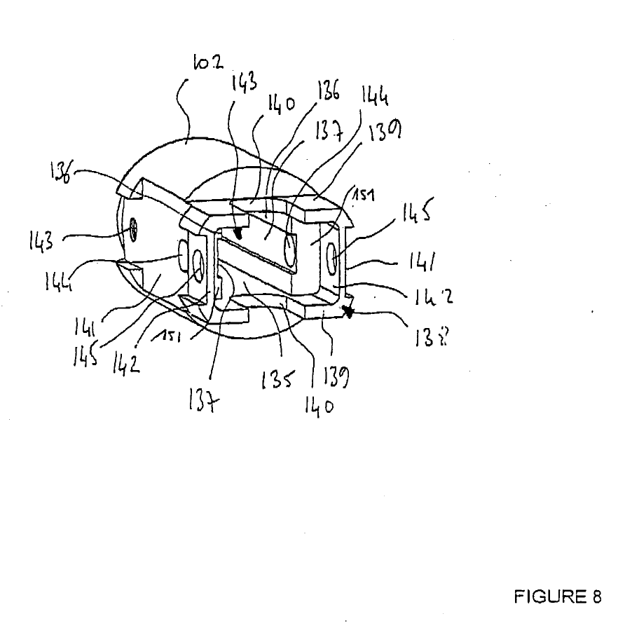

FIGS. 7 and 8 are detailed views representing the free extremity of the main body to be fixed on that of the bone anchoring element.

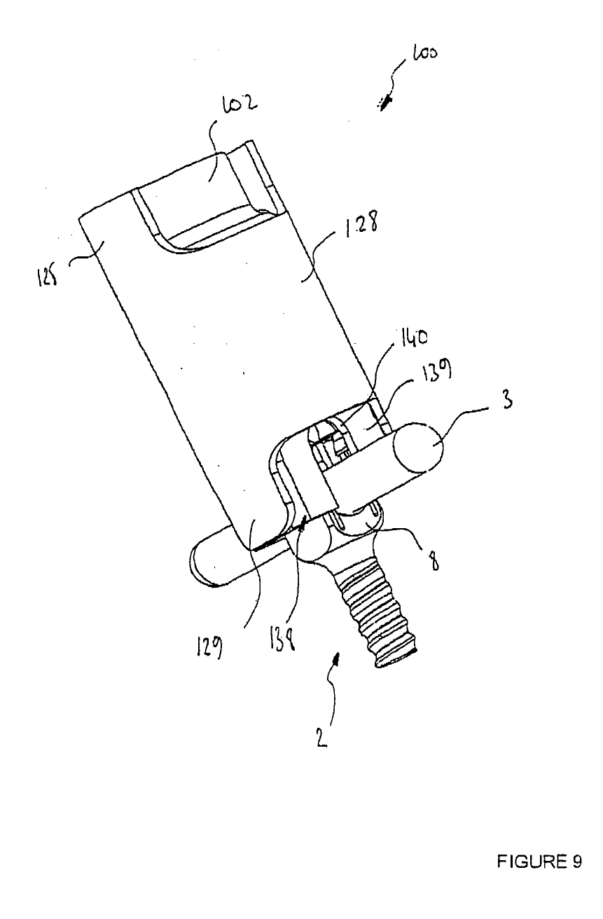

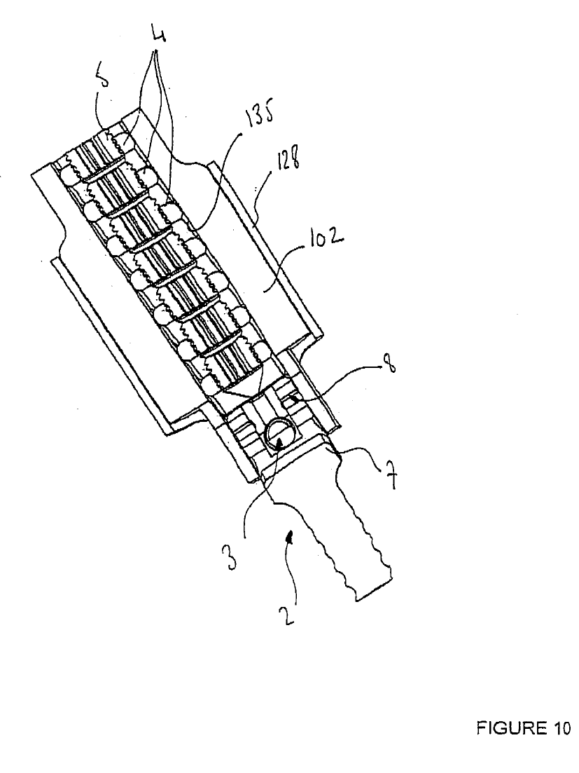

FIGS. 9 and 10 are views showing the different stages of use of the surgical instrument according to the present invention.

In FIG. 1 is shown an example of an embodiment of a spinal implant 1 comprising a bone anchoring element 2 for immobilizing in rotation and in translation a connection rod 3 at each instrumented vertebrae of the vertebral column.

The bone anchoring element 2 cooperates with an immobilizing element 4 allowing the fixation in rotation and in translation of the connection rod 3 through a tightening screw 5.

The bone anchoring element 2 comprises an anchoring part 6 and a receiving part 7 that is comprised of a U-shaped head 8 open in its upper part for cooperating with the connection rod 3 and the immobilizing element 4.

The anchoring part 6 may present either a hook shape, or a threaded profile that is formed or not formed as an integral unit with the receiving part 7 to be fixed on or in the vertebral body of the vertebrae to be instrumented.

The head 8 comprises two vertical walls 9, 10 disposed one facing the other and in parallel planes in order to delimit a first U-shaped central opening 11 carried by the axis XX′ of the connection rod 3 and wherein the bottom 12 presents a profile in a portion of a cylinder.

Each vertical wall 9, 10 is separated, for example, from the bottom 12 of the central opening 11 by a vertical slot 13 giving a certain elasticity to each wall in the direction of the center of the head 8, according to the geometric axis VV′.

The vertical walls 9, 10 respectively comprise at each extremity an engagement part 14, 15 disposed one facing the other and on both sides of the central opening 11.

Each vertical wall 9, 10 comprises on its internal surface and between the engagement parts 14, 15 a vertical housing 16 presenting a profile in a portion of a cylinder provided on both sides of a groove 17 allowing the immobilizing element 4 to be guided during its positioning by means of the surgical instrument 100 in the head 8 of the bone anchoring element 2.

Each vertical wall 9, 10 is pierced between the engagement parts 14, 15 by a hole 18 opening within the central opening 11 and designed to cooperate with the surgical instrument 100 during the introduction of the immobilizing element 4.

The immobilizing element 4 presents an external profile that is roughly parallelepiped shaped comprising, measured parallel to the axis XX′ and in its lower part, a housing 19 presenting a profile in a portion of a cylinder in order to receive and cooperate with the connection rod 3.

The immobilizing element 4 comprises in its middle a tightening screw 5 that opens within the housing 19 to come into contact by pressing against the connection rod 3 to allow its immobilization in rotation and in translation.

The immobilization element 4 comprises in a plane parallel to the axis XX′ tooth-shaped catches 20, 21 separated by a vertical housing 22 laterally bordered by ribs 23 allowing said immobilizing element to be guided during its introduction in the head 8 of the anchoring element 2.

FIGS. 2 and 3 show a surgical instrument 100 allowing the immobilizing element 4 to be positioned and introduced between the walls 9, 10 of the head 8 of the anchoring element 2.

The surgical instrument 100 is comprised of a body 101 comprising a horizontal tube 102 extending from a perforated frame 103 that forms an integral unit measured parallel to axis ZZ′ with a handle 104 called the “fixed handle.”

The fixed handle 104 cooperates with another handle 105 called the “movable handle” that is connected to the first by an axis of rotation 106 and a laminated spring 107 allowing a double acting driving device 108 to be activated under the pressure force.

FIG. 4 illustrates the movable handle 105 comprising above the axis of rotation 106 a head 109 presenting a domed profile 110 from which extends a fork 111 wherein each branch 112 comprises a rounded profile free extremity 113.

FIG. 5 represents the frame 103 of the body 101 that is comprised of two walls, i.e., a vertical wall and an opposite wall 114 and 115 interconnected by perforated upper 116 and lower 117 walls.

The vertical wall 114 is formed as an integral unit with the horizontal tube 102 presenting an internal bore 118 opening within the frame 103, while the opposite wall 115 is pierced by a hole 119 carried by the same main axes as that of the internal bore 118.

The upper wall 116 presents a rectangular-shaped central recess 120, while the lower wall 117 comprises another recess 121 disposed below that of 120. The recess 121 comprises internal shoulders 122 at the fixed handle 104 designed to form a stop.

The upper wall 116 of the frame 103 comprises an extension 123 that extends beyond the vertical wall 115 and in which an oblong groove 124 is pierced.

The surgical instrument 100 comprises a locking tube 125 comprising at one of its extremities fork-shaped branches 127 each provided with notches 126. The tube 125 comprises at the extremity opposite from that in the shape of a fork 127 a cylindrical sleeve 128 extending by parallel tabs 129. The locking tube 125 is constructed around tube 102 so that the free extremities 113 of each branch 112 of the movable handle 105 respectively cooperate with the notches 126 of each branch 127 to allow the sliding of the locking tube 125 around the tube 102 under the action of the movable handle 105.

The surgical instrument 100 comprises a cylindrical plunger 130 that crosses the frame 103 through a hole 119 provided in the vertical wall 115 for sliding in the internal bore 118 of the tube 102 (FIG. 6).

The cylindrical plunger 130 is comprised of a rod comprising at one of its extremities a head 152 in the shape of a domed disk allowing the surgeon to grasp said plunger. The cylindrical plunger 130 has from its head 152 a first cylindrical part 153 that is extended by a second cylindrical part 154 with an outer diameter less than that of the first part.

The driving device 108 of the surgical instrument 100 is comprised of a first plate 131 disposed within frame 103 measured vertically and parallel to axis ZZ′ so as to cooperate with the recesses 120 and 121 of the upper 116 and lower 117 walls to allow it to be guided laterally during its displacements.

The plate 131 is pierced in its middle by a hole opening allowing the passage of the cylindrical plunger 130. The driving device 108 comprises a compression spring 132 that is constructed around the cylindrical plunger 130 so that it can push against the plate 131 and the vertical wall 114 of the frame 103.

The driving device 108 of the surgical instrument 100 is also comprised of a second plate 133 that is positioned according to a direction roughly parallel to axis ZZ′ in order to be guided and held within the groove 124 of the extension 123 of said upper wall 116 of the frame 103.

The second plate 133 is pierced in its middle by a hole opening allowing the passage of the cylindrical plunger 130 in order that the latter crosses, according to a horizontal axis YY′, the frame 103 of the surgical instrument 100.

The driving device 108 comprises a compression spring 134 that is constructed around the cylindrical plunger 130 so as to push against the plate 133 and the vertical wall 115 of the frame 103.

The first plate 131 is provided to push against, under the effect of the spring 132, the domed profile 110 of the head 109 of the movable handle 105 allowing during the activation of the latter the displacement in translation of the cylindrical plunger 130 within the tube 102 and the frame 103.

FIGS. 7 and 8 show the free extremity of tube 102 located opposite from the frame 103 of the body 101 of the surgical instrument 100.

The cylindrical internal bore 118 of the tube 102 is extended at its free extremity by another bore 135 presenting in section a roughly rectangular profile and a shape complementary to that external shape of the immobilizing element 4 of the spinal implant 1.

In fact, the internal bore 135 is provided to receive a certain number of immobilizing elements 4, as for example at least 10 immobilizing elements 4, in order to be able to be presented above each bone anchoring element 2 for their positioning.

Thus, the internal bore 135 comprises, on each parallel and opposite wall 136, a guiding groove 137 provided to each receive the ribs 23 of the corresponding immobilizing element 4.

The cylindrical tube 102 is extended at its free extremity by a sleeve 138 with a roughly rectangular profile wherein each parallel and opposite wall 139 comprises a U-shaped cutout 140 designed to cooperate with the connection rod 3 of the spinal implant 1.

It should be noted that the walls 139 of the sleeve 138 are disposed in planes perpendicular to those containing the walls 136 of the internal bore 135.

It should be noted that the internal profile of the sleeve 138 is measured in dimensions slightly larger than those of the internal bore 135 in order to conform the shoulders 151 forming a stop to the input of the latter.

The cylindrical tube 102 comprises on its external profile and in the planes parallel to those containing the walls 136 of the internal bore 135 flat parts 141 that are extended on each wall 142 of the sleeve 138.

At the cylindrical tube 102, each flat part 141 is pierced by a first hole 143 and a second hole 144 opening within the grooves 137 of the bore 135.

At the walls 142 of the sleeve 138, each flat part 141 is pierced by a third hole opening 145 that is carried by the same main axis as that of the first hole 143 and the second hole 144.

Each first hole 143 presents an internal threading allowing it to cooperate with a tightening screw 146 allowing two elastic strips 147 and 148 to be fixed in each flat part 141.

Each elastic strip 147 is formed as an integral unit with a pin 149 cooperating with the second hole 144 and penetrating within the bore 135 to allow the immobilizing elements 4 to be held within the bore.

Each elastic strip 148 is formed as an integral unit with a pin 150 cooperating with the third hole 145 of the sleeve 138 in order to be able to penetrate within the latter when the strips 148 are deformed by an external pushing force.

One can easily understand from the previous description and from FIGS. 8 and 9 that the operation of the surgical instrument 100 is as follows:

One starts with loading the immobilizing elements 4 within the tube 102 and more particularly within the bore 135. The immobilizing elements 4 are introduced by the lower extremity of the tube 102 at the sleeve 138. During the passage of each immobilizing element 4, the immobilizing pins 149 part due to the elasticity of the strips 147, thus allowing at least 10 immobilizing elements 4 to be introduced within the bore 135 of the tube 102.

One proceeds to the positioning of the surgical instrument 100 on the head 8 of the bone anchoring element 2 of the spinal implant 1 so that the head is positioned in the sleeve 138 and is pushing against the shoulders 151 forming a stop. The orientation of the surgical instrument 100 is defined by the connection rod 3 that must be introduced in the passage of the rod comprised of cutouts 140. In this position, the pins 150 of the elastic strips 148 are positioned facing the holes 18 provided in the head 8 of the bone anchoring element 2.

The surgical instrument 100 is activated by pressing on the movable handle 105 allowing the tube 125 to be displaced in translation through its branches 112 in the direction of the free extremity of the tube 102. During this displacement in translation, the tube 125 pushes by means of the tabs 129 on the elastic strips 148, thus immobilizing the pins 150 in the lateral holes 18 provided in the head 8 of the bone anchoring element 2. This first movement of the handle 105 allows the surgical instrument 100 to be fixed on the bone anchoring element 2.

The introduction of the immobilizing element 4 is done by continuing the pressure on the handle 105 extending its rotation movement and allowing firstly the extremities of each branch 112 of the notches 126 of the tube 125 to be released, which ends the translation of the tube 125 and secondly to come into contact with the plate 131 through the domed profile 110. Due to the thrust exercised on the lower extremity of the plate 131, the latter is positioned straight on the cylindrical plunger 130.

The continuous movement of the handle 105 leading to the plate 131 allows by the straight position of the latter the displacement in translation of the cylindrical plunger 130 within the tube 102.

The cylindrical plunger 130 pushes on the last immobilizing element 4 placed in the internal bore 135 applying a pushing force F to the immobilizing elements 5 assembly contained in the bore 135, allowing the expulsion of the immobilizing element 4 located directly above the head 8 of the bone anchoring element 2 and its introduction in said head 8.

The release of the surgical instrument 100 is obtained by releasing the pressure on the handle 105 which returns through the laminated spring 107 to its position of rest. By returning to its position of rest, the handle 105 leads to the tube 125, due to the notches 126, thus releasing the strips 148 which due to their elasticity separate from the tube 102. The pins 150 leave the holes 18, thus releasing the surgical instrument 100 from the head 8 of the bone anchoring element 2.

Due to the spring 132, the plate 131 returns to its position of rest, stopping on the shoulders 122 of the recess 121 of the frame 103. The cylindrical plunger 130 does not recoil, due to the pressure exercised by the plate 133 that is positioned straight due to the spring 134.

When the last immobilizing element 4 has been introduced, the cylindrical plunger 130 and more particularly the first cylindrical part 153 arrives at the bottom of the bore 118 of the tube 102 while the second cylindrical part 154 is for its part introduced in the bore 135 without penetrating within the sleeve 138. By pushing on the plate 133, the cylindrical plunger 130 is released and may be reassembled. Immobilizing elements 4 may then be reintroduced, as has been previously explained.

In addition, it must be understood that the previous description was given only by way of example and does not limit in any way the scope of the invention, wherein details of the executions described may be replaced by other equivalents without any departure from this scope.

1. A surgical instrument for positioning an immobilizing element (4) in the U-shaped head (8) of a bone anchoring element (2) of a spinal implant (1) for fixing in rotation and in translation a connecting rod (3), characterized in that the instrument comprises a main body (101) comprising a horizontal tube (102) extended by a frame (103) that is formed as an integral unit with a fixed handle (104), a movable handle (105) acting on a driving device (108) guided within the frame (103) and allowing according to the pressure force applied on the movable handle (105) firstly the immobilization of the surgical instrument (100) on the head (8) of the bone anchoring element (2) and secondly the introduction under a pushing force F of an immobilizing element (104), previously positioned and guided within the tube (102), in the head (8) of the bone anchoring element (2).

2. The surgical instrument according to claim 1, characterized in that the movable handle (105) cooperates with the fixed handle (104) through an axis of rotation (106) and a laminated spring (107) allowing the driving device (108) to be activated under a pressure force and to return to its position of rest following release of the pressure force.

3. The surgical instrument according to claim 2, characterized in that the movable handle (105) comprises above the axis of rotation (106) a head (109) presenting a domed profile (110) from which extends a fork (111) wherein each branch (112) comprises a rounded profile free extremity (113).

4. The surgical instrument according to claim 1, characterized in that the frame (103) is comprised of two walls, i.e., a vertical wall and an opposite wall (114, 115) interconnected by upper (116) and lower (117) perforated walls, said vertical wall (114) being formed as an integral unit with the horizontal tube (102) presenting an internal bore (118) opening within the frame (103), while the opposite wall (116) is pierced by a hole (119) carried by the same main axes as that of the internal bore (118), said upper wall (116) presenting a central rectangular-shaped recess (120) and an extension (123) extending beyond the vertical wall (115) and in which an oblong groove (124) is pierced, while the lower wall (117) comprises another recess (121) disposed below that (120), said recess (121) comprising at the fixed handle (104) internal shoulders (122) designed to form a stop.

5. The surgical instrument according to claim 1, characterized in that the instrument comprises a locking tube (125) comprising at one of its extremities branches (127) in the shape of a fork each provided with notches (126) so that the free extremities (113) of each branch (112) of the movable handle (105) respectively cooperate with said notches (126) to allow under the action of the movable handle (105) the sliding of the locking tube (125) around the tube (102) and at the opposite extremity a cylindrical sleeve (128) extended by the parallel tabs (129).

6. The surgical instrument according to claim 1, characterized in that the instrument comprises a cylindrical plunger (130) that crosses the frame (103) through the hole (119) provided in the vertical wall (115) to slide in the internal bore (118) of the tube (102).

7. The surgical instrument according to claim 1, characterized in that the driving device (108) is comprised of a first plate (131) elastically loaded by a spring (132) and disposed within the frame (103) so as to cooperate with the recesses (120, 121) of the upper (116) and lower (117) walls and the cylindrical plunger (130) and of a second plate (133) elastically loaded by a spring (134) and disposed within the groove (124) of the extension (123) of the upper wall (116) so as to cooperate with the cylindrical plunger (130), said first plate (131) being provided to push, under the effect of the spring (132), against the domed profile (110) of the head (109) of the movable handle (105) allowing during activation of the latter the displacement in translation of the cylindrical plunger (130) within the tube (102) due to the straight position of the first plate (131).

8. The surgical instrument according to claim 1, characterized in that the tube (102) comprises in the extension of the cylindrical internal bore (118) and at its free extremity another internal bore (135) wherein the internal profile is complementary to the external profile of the immobilizing element (4) in order to be able to receive and guide the latter before its introduction in the head (8) of the bone anchoring element (2).

9. The surgical instrument according to claim 8, characterized in that the cylindrical tube (102) is extended at its free extremity by a sleeve (138) with a roughly rectangular profile wherein each parallel and opposite wall (139) comprises a U-shaped cutout (140) designed to cooperate with the connection rod (3) of the spinal implant (1).

10. The surgical instrument according to claim 9, characterized in that the internal profile of the sleeve (138) is measured in dimensions slightly larger than those of the internal bore (135) in order to conform the shoulders (151) forming a stop to the input of the bore.

11. The surgical instrument according to claim 10, characterized in that the cylindrical tube (102) comprises on its external profile flat parts (141) that are extended on each wall (142) of the sleeve (138), each flat part (141) being pierced at the tube (102) of the first and second holes (143, 144) opening within the bore (135) and at the walls (142) of the sleeve (138) of a third hole opening (145).

12. The surgical instrument according to claim 11, characterized in that each first hole (143) presents an internal threading allowing the hole to cooperate with a tightening screw (146) for fixing two elastic strips (147, 148) in each flat part (141).

13. The surgical instrument according to claim 12, characterized in that each elastic strip (147) is formed as an integral unit with a pin (149) cooperating with the corresponding second hole (144) and within the bore (135) to allow the immobilizing elements (4) to be held within the bore.

14. The surgical instrument according to claim 12, characterized in that each elastic strip (148) is formed as an integral unit with a pin (150) cooperating with the third hole (145) of the sleeve (138) in order to be able to penetrate within the latter when the strips (148) are deformed by an external pushing force coming from the tabs (129) of the of the locking tube (125) in order to allow the surgical instrument (100) to be fixed on the head (8) of the bone anchoring element (2).

15. The surgical instrument according to claim 8, characterized in that the internal bore (135) of the tube (102) is provided to receive a certain number of immobilizing elements (4).
